(19) United States
(12) Patent Application Publication
(10) Pub. No.: US 2026/0047825 A1
(43) Pub. Date: Feb. 19, 2026

(54) ULTRASOUND IMAGE PROCESSING DEVICE

(71) Applicant: FUJIFILM Corporation, Tokyo (JP)

(72) Inventor: Ryota HASEGAWA, Tokyo (JP)

(73) Assignee: FUJIFILM Corporation, Tokyo (JP)

(21) Appl. No.: 19/296,886

(22) Filed: Aug. 11, 2025

(30) Foreign Application Priority Data
Aug. 13, 2024 (JP) 2024-134815

(51) Int. Cl. A61B 8/00

(52) U.S. Cl. CPC A61B 8/5269

(57) ABSTRACT

A processor removes noise from power data extracted from color flow data, and obtains a gradient at each pixel as blood flow morphology information from the power data after the noise is removed. In addition, the processor performs noise removal on flow velocity data extracted from the color flow data using the blood flow morphology information. In the noise removal, the processor uses, for example, for each pixel, a noise removal filter that considers only a pixel group along a direction perpendicular to the gradient at each pixel.

### CROSS REFERENCE TO RELATED APPLICATION

This application claims priority to Japanese Patent Application No. 2024-134815 filed on Aug. 13, 2024 which is incorporated herein by reference in its entirety including the specification, claims, drawings, and abstract.

### BACKGROUND OF THE DISCLOSURE

### 1. Field of the Disclosure

The present disclosure relates to an ultrasound image processing device, and particularly to removal or reduction of noise in a color Doppler image.

### 2. Description of the Related Art

In a color flow mode of an ultrasound diagnostic apparatus, Doppler signal processing is performed on an echo signal from an inside of a subject detected by an ultrasound probe. Color flow data is generated by the Doppler signal processing, and based on this color flow data, display is performed in various modes such as flow velocity display and power display.

In the flow velocity display, for example, a flow in a direction approaching the probe is color-coded in red, a flow in a direction away from the probe is color-coded in blue, and a magnitude of the flow velocity is represented by a change in hue, saturation, or brightness. In addition, a degree of turbulence (that is, dispersion) in the flow velocity is also displayed using a green component. The flow velocity display is also referred to as color Doppler display or color flow mapping. In addition, in the power display, a strength (that is, the amplitude) of a Doppler signal is represented by a change in hue or brightness.

The color flow data includes noise such as electrical noise, a residual body motion signal, blood flow dropout, and boundary jitter of blood flow. To generate a high-quality display image, it is necessary to remove or reduce such noise.

In the color flow data, power data used for the power display is data having no positive or negative polarity. Therefore, the same noise removal algorithms as those used for grayscale B-mode tomographic images having no polarity can be used for noise removal of the power data. Although advanced noise removal algorithms using a non-linear filter, a multi-resolution filter, or the like are used for the B-mode tomographic images, such advanced noise removal algorithms can also be used for the power display.

Contrary to this, since flow velocity data used for the flow velocity display has positive and negative polarities, advanced noise removal algorithms using a non-linear filter, a multi-resolution filter, or the like cannot be directly applied. It is also conceivable to use such advanced noise removal algorithms after modification to account for polarity, but a calculational load for this is very large. Therefore, in the related art, simple smoothing processing using an averaging filter, a Gaussian filter, or the like has been used for noise removal for the flow velocity display. With such simple smoothing processing, boundaries of the blood flow in the flow velocity display tend to become blurred.

As a technology in the related art regarding noise removal in an ultrasound diagnostic apparatus, there is an apparatus disclosed in JP2014/069374A. The apparatus in the related art addresses a problem in that, in a case where removal of noise within a heart chamber is performed on an ultrasound image of a heart, a signal from a myocardial region is also removed, and a boundary between the heart chamber and a myocardium is blurred, whereas in a case where the signal from the myocardial region is enhanced, noise within the heart chamber is also enhanced. This apparatus comprises: a signal enhancement processing unit that performs signal enhancement processing on medical image data; a noise removal unit that performs noise removal processing on the medical image data; a first signal compression processing unit that compresses the medical image data subjected to the signal enhancement processing and the noise removal processing; a second signal compression processing unit that compresses the medical image data; and a composition processing unit that combines the medical image data compressed by the first signal compression processing unit and the medical image data compressed by the second signal compression processing unit.

In addition, an ultrasound diagnostic apparatus disclosed in JP1995-308318A (JP-H07-308318A) comprises: a transmission unit that transmits an ultrasound beam into a living body; a reception unit that receives a reflected echo signal from the living body; a blood flow signal processing unit that performs Doppler signal processing on the reflected echo signal to generate color flow data including an average flow velocity, a power value, and a velocity variance; an averaging processing unit that performs averaging processing on at least the power value of the color flow data; a comparison unit that compares the color flow data subjected to the averaging processing by the averaging processing unit with a set threshold; and a display unit that displays the color flow data based on a comparison result of the comparison unit.

### SUMMARY OF THE DISCLOSURE

An object of the present disclosure is to prevent or reduce blurring of boundaries of blood flow in display of flow velocity data.

In one aspect, an ultrasound image processing device according to the present disclosure comprises: a processor, in which the processor acquires a flow velocity value and a power value of each pixel in color flow data, obtains morphology information indicating a morphology of blood flow based on the power value of each pixel, and executes noise removal on the flow velocity value of each pixel based on the morphology information.

In a certain aspect, the morphology information may be a gradient of the power value at each pixel, the noise removal for the flow velocity value may include, for each pixel, calculation of obtaining, from flow velocity values of a pixel and surrounding pixels of the pixel, a flow velocity value after noise removal of the pixel, and in the calculation, in a case where a direction perpendicular to the gradient at the pixel is referred to as a first direction, a greater weight may be assigned to a flow velocity value of a pixel among the surrounding pixels on a side in the first direction with respect to the pixel compared to a weight assigned to a flow velocity value of a pixel that is not on the side in the first direction with respect to the pixel.

In another aspect, the morphology information may be a contour of the blood flow, the noise removal for the flow velocity value may be processing of calculating, for each pixel, from flow velocity values of a pixel and surrounding pixels of the pixel, a flow velocity value after noise removal of the pixel, and in the calculation, in a case where a direction in which the contour extends in a vicinity of the pixel is referred to as a second direction, a greater weight may be assigned to a flow velocity value of a pixel among the surrounding pixels on a side in the second direction with respect to the pixel compared to a weight assigned to a flow velocity value of a pixel that is not on the side in the second direction with respect to the pixel.

In still another aspect, the processor may execute noise removal on the acquired power value of each pixel, and the morphology information may be calculated based on the power value after the noise removal.

In addition, in another aspect, a program according to the present disclosure may cause a computer to execute a process comprising: acquiring a flow velocity value and a power value of each pixel in color flow data; obtaining morphology information indicating a morphology of blood flow based on the power value of each pixel; and executing noise removal on the flow velocity value of each pixel based on the morphology information.

According to the present disclosure, it is possible to prevent or reduce blurring of boundaries of blood flow in display of flow velocity data.

### BRIEF DESCRIPTION OF THE DRAWINGS

FIG. 1 is a diagram showing an example of a hardware configuration of a computer that executes processing of an embodiment.

FIG. 2 is a diagram showing an example of a processing flow of flow velocity (that is, color Doppler) display in the embodiment.

FIG. 3 is a diagram showing an example of a procedure of noise removal for flow velocity data.

FIG. 4 is a diagram showing an example of a kernel of a noise removal filter held by an ultrasound image processing device.

FIG. 5 is a diagram showing another example of the procedure of the noise removal for the flow velocity data.

### DESCRIPTION OF THE PREFERRED EMBODIMENTS

Hereinafter, embodiments of the present disclosure will be described with reference to the drawings. An ultrasound image processing device in the present embodiment generates an ultrasound diagnostic image displayed by an ultrasound diagnostic apparatus. An example of the ultrasound diagnostic image generated by the ultrasound image processing device is a color Doppler image for flow velocity display.

Processing for image generation performed by the ultrasound image processing device of the present embodiment is executed by, for example, a computer. In one example, the computer is built into the ultrasound diagnostic apparatus. In this example, the ultrasound image processing device of the present embodiment is the ultrasound diagnostic apparatus itself or an image processing system into which the ultrasound diagnostic apparatus is built.

In addition, in another example, the processing for the image generation in the present embodiment may be executed by an external computer outside the ultrasound diagnostic apparatus. In this example, the external computer is connected to the ultrasound diagnostic apparatus via a communication path such as a data communication network. The external computer in this example may be a single computer, or may be made up of a plurality of computers that cooperate with each other through communication via a data communication network to implement the processing.

In addition, in another example, the processing may be executed by cooperation between the computer built into the ultrasound diagnostic apparatus and the external computer connected to the ultrasound diagnostic apparatus.

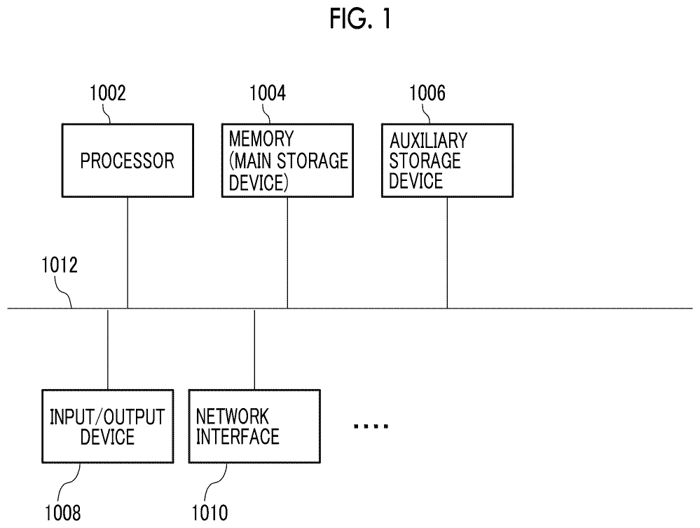

FIG. 1 shows an example of a hardware configuration of a computer built into the ultrasound diagnostic apparatus or connected to the ultrasound diagnostic apparatus. The computer shown in the figure has a circuit configuration in which a processor 1002, a memory (main storage device) 1004 such as a random-access memory (RAM), a controller that controls an auxiliary storage device 1006 which is a non-volatile storage device such as a flash memory, a solid-state drive (SSD), or a hard disk drive (HDD), an interface with various input/output devices 1008, a network interface 1010 that performs control for connection to a network such as a local area network, and the like are connected via a data transmission path such as a bus 1012. For example, a program describing the processing of the present embodiment is installed on the computer and is stored in the auxiliary storage device 1006. The program stored in the auxiliary storage device 1006 is executed by the processor 1002 using the memory 1004, thereby implementing the ultrasound image processing device in the present embodiment.

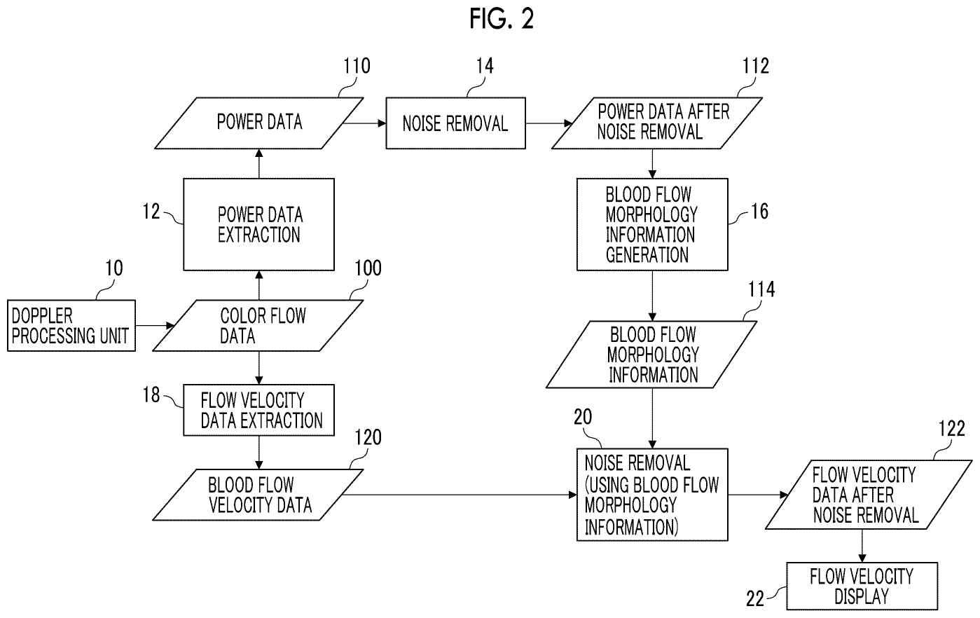

FIG. 2 shows an example of a flow of processing executed by the processor 1002 of the ultrasound image processing device according to the present embodiment. Hereinafter, this flow will be described.

A Doppler processing unit 10 generates color flow data 100 by executing known Doppler signal processing on an echo signal detected by an ultrasound probe of the ultrasound diagnostic apparatus. The color flow data 100 includes information such as a flow velocity, a power value (that is, an amplitude of a Doppler signal), and a variance of the flow velocity at each position in a subject. The term “position” here corresponds to a pixel in an ultrasound image representing an inside of the subject. In a case where the color flow data 100 is composed of information for each pixel in the ultrasound image, the color flow data 100 includes, for each pixel, information such as a flow velocity, a power value, and a variance of the pixel. The Doppler processing unit 10 may remove a low-speed signal from a surrounding tissue or the like using a moving target indicator (MTI) filter or the like to obtain data on blood flow components.

The processor 1002 of the ultrasound image processing device extracts power data 110 indicating the power value at each position (that is, each pixel) from the color flow data 100 (block 12). The extracted power data 110 includes information on the power value at each position.

Next, the processor 1002 executes noise removal on the power data 110 (block 14). Since the power value at each position included in the power data 110 is a non-negative value, any of various methods used for noise removal of B-mode tomographic images may be used for the noise removal. Through this noise removal, power data 112 after the noise removal is obtained.

The processor 1002 generates blood flow morphology information 114 indicating a morphology of blood flow from the power data 112 after the noise removal (block 16). The morphology of the blood flow referred to here is a form or shape of blood flow inside a blood vessel. For example, a morphology or shape of a boundary of the blood flow (that is, a boundary between the blood flow and the surrounding tissue) is a representative example. In addition, another example of the blood flow morphology information is a gradient at each position of the power data 112.

In addition, the processor 1002 extracts flow velocity data 120 indicating a flow velocity value at each position (that is, each pixel) from the color flow data 100 (block 18) in parallel with the extraction of the power data 110 (block 12). The extracted flow velocity data 120 includes information on the flow velocity at each position. In a case where a signal from a surrounding tissue outside the blood flow is removed by the MTI filter, the flow velocity at each position in the surrounding tissue is approximately 0.

Next, the processor 1002 performs noise removal calculation on the flow velocity data 120 (block 20). The noise removal calculation is performed with reference to the blood flow morphology information 114. Processing performed in the noise removal (block 20) will be described in detail later with reference to examples. Through this noise removal, flow velocity data 122 after the noise removal is obtained.

The processor 1002 generates an image (that is, a color Doppler image) for the flow velocity display using the flow velocity value at each position (that is, each pixel) in the flow velocity data 122 after the noise removal, and displays the image on a display device (not shown) (block 22).

According to the processing shown in FIG. 2, by performing noise removal on the flow velocity data 120 with reference to the blood flow morphology information 114, blurring of the flow velocity value at a position near the boundary of the blood flow due to an influence of a velocity value of the surrounding tissue is prevented.

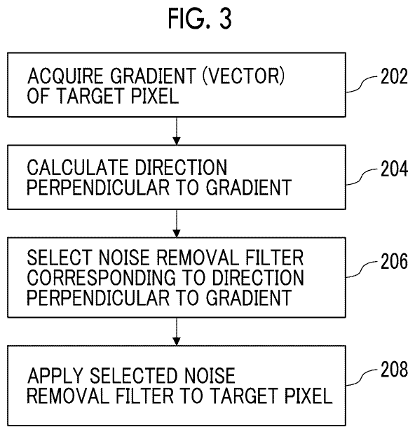

Next, an example of processing contents of the noise removal (block 20) performed on the flow velocity data in the processing flow shown in FIG. 2 will be described with reference to FIG. 3.

In this example, in block 16 of the processing flow of FIG. 2, a gradient value of each pixel is calculated for the power data 112, which is a scalar field, as the blood flow morphology information. In a processing procedure of FIG. 3, the gradient of each pixel is referenced.

The processor 1002 executes the procedure of FIG. 3 for each pixel included in the flow velocity data 120, with the pixel as a target pixel.

In this procedure, the processor 1002 first acquires the value of the gradient of the target pixel from the blood flow morphology information obtained in block 16 (block 202). Next, the processor 1002 calculates a direction perpendicular to the gradient acquired in block 202 (block 204).

Next, the processor 1002 selects a noise removal filter corresponding to the direction calculated in block 204 (block 206). That is, the ultrasound image processing device holds a plurality of noise removal filters each corresponding to a different direction, and the processor 1002 selects a direction closest to the direction calculated in block 204 from among the plurality of filters.

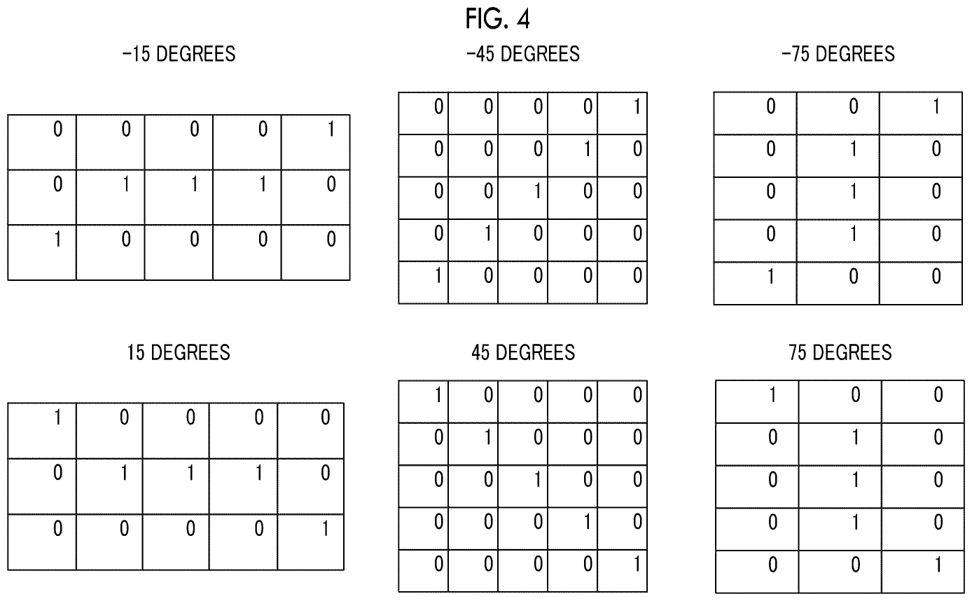

FIG. 4 shows an example of a kernel of the noise removal filter held by the ultrasound image processing device. In this example, the ultrasound image processing device holds kernels for six directions with different angles in increments of 30 degrees, such as −75 degrees, −45 degrees, −15 degrees, +15 degrees, +45 degrees, and +75 degrees. Each of individual cells constituting the illustrated kernel corresponds to a pixel in the ultrasound image, and a central cell corresponds to the target pixel that is a target of a noise removal operation. In addition, in the illustrated kernel, a value of each cell is a binary value of either 0 or 1. In this example, a pixel corresponding to the cell having a value of 1 is a valid pixel, that is, a pixel referenced in the noise removal calculation. In addition, a pixel having a value of 0 is an invalid pixel that is not referenced in the calculation. For example, in a case where the −45-degree kernel is used, in the filter processing, a filter output is calculated by referencing a group of pixels arranged along a 45-degree diagonal line slanting upward to the right, with the target pixel at the center.

For example, in a case where a median filter is used as the noise removal filter, a median value among pixel values (that is, the flow velocity values in this example) of a plurality of valid pixels indicated by the kernel is obtained as the filter output. In a case where an averaging filter is used, an average value of the pixel values of the valid pixels is obtained as the filter output. In this example, values of the invalid pixels are not reflected in the filter output.

In the example of FIG. 4, the values of the cells corresponding to the valid pixels in the kernel are all 1, but this is merely an example. As another example of the kernel, a kernel in which a value differs for each cell corresponding to a valid pixel may be used. By assigning a different value to each cell corresponding to a valid pixel, a filter that performs noise removal by weighted averaging can be constructed. For example, a kernel of a Gaussian filter can be constructed by setting the value of the cell corresponding to the target pixel to be the maximum value and decreasing the values of the other cells according to a Gaussian distribution as the distance from the cell increases.

In addition, in the above example, a median filter, a simple averaging filter, and a weighted averaging filter have been presented as examples, but other types of filters may be used as the noise removal filter.

In addition, in the above example, the value of the cell corresponding to the invalid pixel is set to 0, but this is merely an example. Alternatively, the value of the cell corresponding to the invalid pixel may be set to a positive value that is significantly smaller than the value of the cell corresponding to the valid pixel. Even in this case, the value of the filter output is determined primarily by the value of the valid pixel, and the influence of the invalid pixel is negligible or extremely small.

In block 206, among the plurality of prepared filters (that is, kernels), a filter in a direction closest to the direction calculated in block 204 is selected.

Then, the processor 1002 applies the selected filter to the target pixel to obtain the filter output value corresponding to the target pixel (block 208). That is, the processor 1002 aligns, for example, the selected filter with the target pixel in the image represented by the flow velocity data. Then, the processor 1002 calculates the filter output value by performing a filter operation on the image using the value of each cell indicated by the filter. Contents of the filter operation depend on the type of filter used as the noise removal filter (for example, a median filter or an averaging filter).

As described above, in the present embodiment, the noise removal for the flow velocity value of the target pixel is performed along a direction perpendicular to the gradient of the power value at the target pixel.

The noise removal includes calculation (for example, the filter operation described above) of obtaining the flow velocity value after the noise removal of the target pixel from the flow velocity values of the target pixel and surrounding pixels of the target pixel. Here, the surrounding pixels of the target pixel are pixels within a predetermined range (for example, a distance in units of pixels) as viewed from the target pixel. For example, in a case where the kernel described above is aligned with the target pixel, pixels at locations corresponding to each cell in the kernel are examples of the “surrounding pixels” described above. In addition, in the calculation of obtaining the flow velocity value after the noise removal, greater weights are assigned to the flow velocity values of the pixels located on a side in a first direction with respect to the target pixel among the “surrounding pixels”, compared to weights assigned to flow velocity values of pixels not on the side in the first direction with respect to the target pixel. Here, the first direction is a direction perpendicular to the gradient at the target pixel. The pixel on the side in the first direction with respect to the target pixel is not limited to a pixel that is strictly in the first direction with respect to the target pixel, and may be a pixel within a predetermined range (for example, at a predetermined distance or a predetermined angle) from a position along the first direction. In addition, the weight assigned to the pixel that is not on the side in the first direction may be 0 as in the kernel illustrated in FIG. 4, or may be a small positive value close to 0.

By the above-described processing procedure, noise removal is performed by referencing a group of pixels arranged along the morphology of the blood flow, that is, in this example, in a direction perpendicular to the gradient of the power value. As a result, blurring of the boundaries of the blood flow or the like, which has occurred in the simple filter processing in the related art, is suppressed or reduced.

In addition, in the above-described processing procedure, the blood flow morphology information is obtained based on the power data after noise removal, so that the influence of noise included in the power data can be reduced, and smooth blood flow morphology information can be obtained.

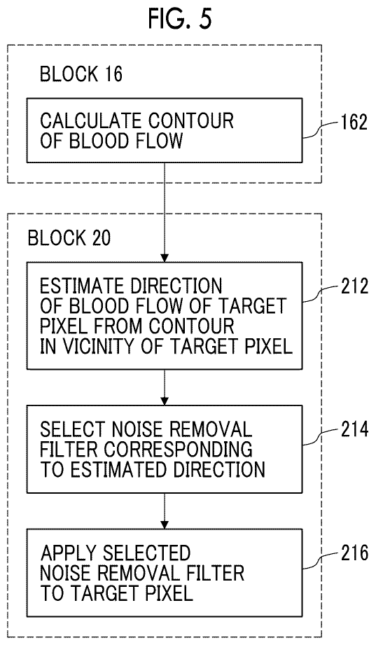

Next, another example of the processing of calculating the blood flow morphology information (block 16) and the noise removal (block 20) will be described with reference to FIG. 5.

In this example, a contour of the blood flow is obtained as the blood flow morphology information. It is well known that the contour of the blood flow appears distinctly in power Doppler display that displays power data. Information on the contour is used as the blood flow morphology information.

In the procedure of FIG. 5, the processor 1002 obtains the contour of the blood flow as the blood flow morphology information from the power data 112 after the noise removal (block 162). In the calculation of block 162, for example, the contour may be detected by applying an edge detection filter to the image represented by the power data 112. Alternatively, the power data 112 may first be binarized to be divided into a blood flow region and other regions, and then an edge detection filter may be applied to detect the contour.

Next, the processor 1002 executes noise removal on the flow velocity data 120 by using information on the contour of the blood flow obtained in block 162 (block 20). In this block 20, the processor 1002 executes the procedure of blocks 212 to 216 of FIG. 5 for each pixel included in the flow velocity data 120, with the pixel as the target pixel.

First, in block 212, the processor 1002 estimates a direction of the blood flow at the target pixel from a contour region in the vicinity of the target pixel in the contour of the blood flow obtained in block 162. In this estimation, for example, the processor 1002 obtains a point closest to the target pixel among points on the contour of the blood flow, and obtains a direction of a tangent to the contour line at the point. Then, the processor 1002 estimates the direction of the tangent as the direction of the blood flow at the target pixel. As another example, the processor 1002 may estimate a direction in which a contour line of a predetermined range (for example, several pixels) in the vicinity of a point closest to the target pixel in the contour of the blood flow extends as the direction of the blood flow.

Next, the processor 1002 selects, from among the noise removal filters (that is, kernels) held by the ultrasound image processing device, a noise removal filter corresponding to the direction estimated in block 212, for example, a noise removal filter of which a direction is closest to that direction (block 214). Then, the processor 1002 applies the selected filter to the target pixel to obtain the filter output value corresponding to the target pixel (block 216). The processing in blocks 214 and 216 may be the same as the processing in blocks 206 and 208 of the procedure of FIG. 3.

As described above, in the example of FIG. 5, the noise removal for the flow velocity value of the target pixel is performed along the direction in which a region of the contour of the blood flow in the vicinity of the target pixel extends.

The noise removal includes calculation of obtaining the flow velocity value after the noise removal of the target pixel from the flow velocity values of the target pixel and surrounding pixels of the target pixel. Here, the surrounding pixels of the target pixel are pixels within a predetermined range as viewed from the target pixel. For example, in a case where the kernel described above is aligned with the target pixel, pixels at locations corresponding to each cell in the kernel are examples of the “surrounding pixels” described above. In addition, in the calculation of obtaining the flow velocity value after the noise removal, greater weights are assigned to the flow velocity values of the pixels located on a side in a second direction with respect to the target pixel among the “surrounding pixels”, compared to weights assigned to flow velocity values of pixels that are not on the side in the second direction with respect to the target pixel. Here, the second direction is a direction in which the region of the contour of the blood flow in the vicinity of the target pixel extends. The pixel on the side in the second direction with respect to the target pixel is not limited to a pixel that is strictly in the second direction with respect to the target pixel, and may be a pixel within a predetermined range (for example, at a predetermined distance or a predetermined angle) from a position along the second direction. In addition, the weight assigned to the pixel that is not on the side in the second direction may be 0 as in the kernel illustrated in FIG. 4, or may be a small positive value close to 0.

Although the embodiments of the present disclosure have been described above, they are merely examples for describing the present disclosure. For example, in the above-described embodiments, the gradient of the power data or the contour of the blood flow is used as the blood flow morphology information, but this is merely an example. As another example, contour lines of the power data may be obtained using a known contour line calculation algorithm, and information on the contour line may be used as the blood flow morphology information. In this example, a contour line is a line connecting pixels that have the same power value. In this case, the processor 1002 performs the noise removal by applying a kernel corresponding to a direction in which the contour line passing through the target pixel extends (for example, a tangent direction of the contour line in the target pixel) to the target pixel.

In the present embodiment, each process is executed by any computer. In addition, any computer may execute such processes using a processor as hardware, a program as software, or a combination of both. In such a case, the processor may function as each unit or each means that executes various processes in the present embodiment. Furthermore, an execution order of the processes by the processor is not limited to the order described above and may be changed as appropriate. Any computer may be a general-purpose computer, a dedicated computer for a specific use, a workstation, or another system capable of executing each process.

The processor may be configured by one or a plurality of types of hardware, and the type of hardware is not limited. For example, the processor can be configured by hardware such as a central processing unit (CPU), a micro processing unit (MPU), a programmable logic device such as a field-programmable gate array (FPGA), a dedicated circuit for executing specific processing such as an application-specific integrated circuit (ASIC), a graphics processing unit (GPU), or a neural processing unit (NPU). In addition, the type of hardware may be a combination of different types of hardware. In a case where the plurality of types of hardware are configured to execute one or a plurality of processes of a certain processor, the plurality of types of hardware may be present in devices physically separated from each other, or may be present in the same device. In any of the embodiments, the procedure of each processing by the processor is not limited to the above procedure and may be changed as appropriate. The hardware is configured by an electric circuit (circuitry) in which circuit elements such as semiconductor elements are combined.

Furthermore, the program may be software such as firmware or a microcode. In addition, the program may be, for example, a group of program modules, and each function thereof may be implemented by a processor configured to execute the corresponding function. The program may be a program code or a plurality of code segments stored in one or a plurality of non-transitory computer-readable media (for example, storage media or other types of storage). The program may be distributed and stored in the plurality of non-transitory computer-readable media existing in devices physically separated from each other. The program code or code segment may represent any combination of a procedure, a function, a subprogram, a routine, a subroutine, a module, a software package, a class, or an instruction, a data structure, or a program statement. The program code or code segment may be connected to another code segment or a hardware circuit by transmitting and receiving information, data, an argument, a parameter, or a content of a memory.

In addition, the hardware constituting the processor may include a digital signal processing circuit or an analog signal processing circuit.

What is claimed is:

1. An ultrasound image processing device comprising:
a processor,
wherein the processor
acquires a flow velocity value and a power value of each pixel in color flow data,
obtains morphology information indicating a morphology of blood flow based on the power value of each pixel, and
executes noise removal on the flow velocity value of each pixel based on the morphology information.

2. The ultrasound image processing device according to claim 1,
wherein the morphology information is a gradient of the power value at each pixel, and
the noise removal for the flow velocity value includes, for each pixel, calculation of obtaining, from flow velocity values of a pixel and a surrounding pixel of the pixel, a flow velocity value after noise removal of the pixel, and in the calculation, in a case where a direction perpendicular to the gradient at the pixel is referred to as a first direction, a greater weight is assigned to a flow velocity value of a pixel among the surrounding pixels on a side in the first direction with respect to the pixel compared to a weight assigned to a flow velocity value of a pixel that is not on the side in the first direction with respect to the pixel.

3. The ultrasound image processing device according to claim 1,
wherein the morphology information is a contour of the blood flow, and
the noise removal for the flow velocity value is processing of calculating, for each pixel, from flow velocity values of a pixel and a surrounding pixel of the pixel, a flow velocity value after noise removal of the pixel, and in the calculation, in a case where a direction in which the contour extends in a vicinity of the pixel is referred to as a second direction, a greater weight is assigned to a flow velocity value of a pixel among the surrounding pixels on a side in the second direction with respect to the pixel compared to a weight assigned to a flow velocity value of a pixel that is not on the side in the second direction with respect to the pixel.

4. The ultrasound image processing device according to claim 1,
wherein the processor executes noise removal on the acquired power value of each pixel, and
the morphology information is calculated based on the power value after the noise removal.

5. The ultrasound image processing device according to claim 2,
wherein the processor executes noise removal on the acquired power value of each pixel, and
the morphology information is calculated based on the power value after the noise removal.

6. The ultrasound image processing device according to claim 3,
wherein the processor executes noise removal on the acquired power value of each pixel, and
the morphology information is calculated based on the power value after the noise removal.

7. A program causing a computer to execute a process comprising:
acquiring a flow velocity value and a power value of each pixel in color flow data;
obtaining morphology information indicating a morphology of blood flow based on the power value of each pixel; and
executing noise removal on the flow velocity value of each pixel based on the morphology information.
